(19) United States
(12) Patent Application Publication
(10) Pub. No.: US 2023/0145406 A1
(43) Pub. Date: May 11, 2023

(54) DIVALENT METAL-CONTAINING BIOMATERIALS AND METHODS OF USE

(71) Applicant: University of Oregon, Eugene, OR (US)

(72) Inventors: Balamurali Ambati, Eugene, OR (US); Sangeetha Ravi Kumar, Eugene, OR (US); Kaleb Roach, Eugene, OR (US)

(73) Assignee: University of Oregon, Eugene, OR (US)

(21) Appl. No.: 17/985,058

(22) Filed: Nov. 10, 2022

Related U.S. Application Data
(60) Provisional application No. 63/278,056, filed on Nov. 10, 2021.

(51) Int. Cl. A61K 9/00; A61F 9/007; A61K 31/715; A61K 47/34; A61P 27/02

(52) U.S. Cl. CPC A61K 9/0024; A61F 9/00772; A61K 31/715; A61K 9/0051; A61K 47/34; A61P 27/02

(57) ABSTRACT

Biocompatible materials including a biodegradable hydrogel and an active agent including a divalent metal salt are provided. Methods of making the biocompatible materials and methods of use, for example for treating an eye disease or disorder, such as keratoconus, are also provided.

### CROSS REFERENCE TO RELATED APPLICATION

This application claims the benefit of U.S. Provisional Application No. 63/278,056, filed Nov. 10, 2021, which is incorporated herein by reference in its entirety.

### FIELD

This disclosure relates to divalent metal-containing biomaterials and methods of use, particularly for treating keratoconus.

### BACKGROUND

Keratoconus is an ocular pathology that is characterized by weakening of collagen cross linkages in the cornea, resulting in progressive stromal thinning and ectasia in the cornea. In the initial stages, contact lens and implants such as intrastromal corneal rings segments may be helpful, but are rendered ineffective in advanced stages, leaving corneal transplantation as the only option. Collagen cross linkages in the cornea and sclera are catalyzed by the copper containing enzyme, lysyl oxidase. Previous studies have reported a decrease in lysyl oxidase activity in the corneas of keratoconus patients.

### SUMMARY

There is a need for improved methods for treating keratoconus. Topical copper containing eye drops have been found to significantly upregulate lysyl oxidase activity and collagen cross linkages. Punctal plugs have advantages of delivering drugs directly to the eye, as well as reducing dry eye by retaining tears and keeping the eye moist. In addition, they overcome the discomfort of eyedrops and provide for consistent compliance and a targeted approach.

Provided herein are divalent metal-containing biomaterials that are implantable in the eye (such as plugs or films). The biomaterials gradually release the divalent metal (such as copper) over time (such as about 3-6 months) and in some examples are used for treating eye disorders, such as keratoconus.

Provided herein are biocompatible materials that include a biodegradable hydrogel and a therapeutically effective amount of an active agent including a divalent metal salt, wherein the active agent is incorporated in or is in association with the hydrogel. In some embodiments, the biodegradable hydrogel includes polyethylene glycol or polycaprolactone, for example, polyethylene glycol diacrylate or polycaprolactone diacrylate.

In some embodiments, the active agent includes a divalent metal salt, such as a copper salt, a magnesium salt, or an iron salt. In some examples, the divalent metal salt is a copper salt, such as copper sulfate, copper citrate, copper acetate, copper nicotinate, copper carbonate, copper phosphate, copper dihydrogen phosphate, copper asprinate, copper naproxen, copper histidine, copper stearate, copper oleate, copper chloride, copper oxide, copper lactate, copper dilactate, copper hydroxide phosphate, or copper threonate. In one example, the copper salt is copper dilactate. In some embodiments, the biocompatible material includes about 1.5 to about 40 wt % of the divalent metal, such as about 1.5 to about 40 wt % copper. In some examples, the divalent metal salt is released from the biocompatible material over time, such as wherein about 0.5 μg to about 1 μg of divalent metal (for example, copper) is released per day.

In some embodiments, the biocompatible material is in the form of an implantable material, such as a punctal plug or film. In some examples, the material biodegrades or resorbs over at least 3 months when implanted in an eye.

Also provided are methods of making the disclosed biocompatible materials. In some embodiments, the methods include mixing a pre-polymer (such as polyethylene glycol or polycaprolactone pre-polymer, for example, PEGDA or PCL-DA) with a divalent metal salt solution, sonicating the mixture of pre-polymer and divalent metal salt solution, adding a photoinitiator, and curing under UV light to form a divalent metal salt-containing material. In some examples, the methods include placing the mixture of pre-polymer, divalent metal solution, and photoinitiator in a mold prior to curing.

In some embodiments, the divalent metal salt solution is prepared by sonicating a solution of the divalent metal salt, incubating the sonicated solution at 37° C., and cooling the solution to room temperature. In some examples, the divalent metal salt solution is sonicated for a total of about 15 to 120 minutes. In some examples, the sonicated solution is incubated at 37° C. for about 6 to about 24 hours. In some examples, the divalent metal salt solution is a copper salt solution. In some examples, the copper salt is copper sulfate, copper citrate, copper acetate, copper nicotinate, copper carbonate, copper phosphate, copper dihydrogen phosphate, copper asprinate, copper naproxen, copper histidine, copper stearate, copper oleate, copper chloride, copper oxide, copper lactate, copper dilactate, copper hydroxide phosphate, or copper threonate. In one example, the copper salt is copper dilactate.

Also provided are methods of treating an eye disease or disorder in a subject using the disclosed biocompatible materials. In some examples, the subject has keratoconus, myopia, presbyopia, corneal ulcers, corneal marginal degenerations, astigmatism, scleromalacia, or corneal ectasia. In some embodiments, the methods include contacting the eye of the subject with a disclosed biocompatible material. In some examples, the biocompatible material is a punctal plug and is placed in contact with a tear duct of the subject. In other examples, the biocompatible material is a film and is placed in contact with a fornix of an eye of the subject. In additional embodiments, the methods further include administering to the subject one or more additional therapies for treatment of the eye disease or disorder. In some examples, one or more symptoms of the disorder in the subject are improved, for example compared to a control (such as an untreated subject or eye, or the same subject prior to treatment).

The foregoing and other features of the disclosure will become more apparent from the following detailed description, which proceeds with reference to the accompanying FIGURE.

### BRIEF DESCRIPTION OF THE DRAWINGS

FIGS. 1A-1E illustrate various embodiments of the disclosure. FIG. 1A is a POC native polymer. FIG. 1B shows POC with CuSO₄. FIG. 1C is a magnified image of PEG based polymer with cupric dilactate. FIG. 1D shows a 0.3 mm PEG based polymer with cupric dilactate. FIG. 1E is a 0.4 mm commercial punctal plug used for dry eye disease.

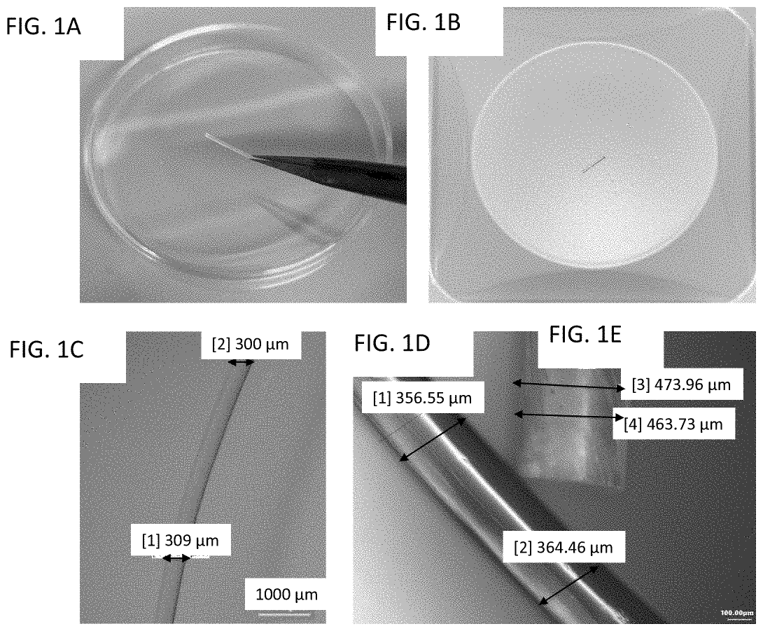

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. As used herein, the term “comprises” means “includes.” It is further to be understood that any and all base sizes or amino acid sizes, and all molecular weight or molecular mass values, are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Active agent: An agent that can have a beneficial effect on a subject when administered. In some examples, an active agent is a compound that can improve one or more signs or symptoms of keratoconus in a subject, such as a divalent metal salt-containing compound. In one example, an active agent is a copper-containing compound.

Biocompatible: The property of a biomaterial or device having the ability to perform its desired function (for example, with respect to a medical therapy), without eliciting any undesirable local or systemic effects (such as undesirable immune responses) in a subject. A biocompatible material or device ideally also generates a beneficial effect or cellular or tissue response. In some examples, biocompatible refers to a material or device that is enzymatically or chemically degraded in vivo into simpler chemical species (“biodegradable”). A biocompatible material, device, or system includes synthetic or natural material used to function in close contact with living tissue.

Contacting: Placement in direct physical association; includes both in solid and liquid form.

Fornix: The fornix conjunctiva (“fornix”) is the loose soft tissue lying at the junction between the palpebral conjunctiva (covering the inner surface of the eyelid) and the bulbar conjunctiva (covering the globe). Each eye has two fornices, the superior (“upper”) fornix and the inferior (“lower”) fornix.

Inhibiting or treating a disease: Inhibiting a disease or disorder refers to inhibiting the full development of a disease or disorder or lessening the physiological effects of the disease process. In several examples, inhibiting or treating a disease or disorder refers to lessening symptoms of keratoconus. For example, treatment can lessen a sign or symptom of keratoconus. “Treatment” refers to a therapeutic intervention that ameliorates a sign or symptom of a disease or pathological condition related to the disease. The treatment may also prevent or delay the development of one or more symptoms of the disease or disorder. “Ameliorating” refers to the reduction in the number or severity of signs or symptoms of a disease or disorder, such as keratoconus.

Keratoconus: A disorder characterized by thinning of the cornea and bulging outward into a cone shape, scarring, and eventual rupture of Descemet's membrane. Symptoms include blurred or distorted vision, sensitivity to glare and light, irregular astigmatism, and corneal scarring. Cross-liking between collagen and elastin fibrils maintains the biomechanical properties of the cornea. Keratoconus is characterized by inadequate collagen cross-linking. The cause of keratoconus is not known, but it has been associated with connective tissue disorders (such as Marfan syndrome or Ehler-Danlos syndrome), eye allergies, and excessive eye rubbing. There may also be a genetic component, as about 10% of individuals with keratoconus have a parent with keratoconus.

Lysyl oxidase (LOX): A copper-dependent amine oxidase that functions in cross-liking of collagens and elastin. LOX activity has been demonstrated to be reduced in keratoconic corneas.

Punctal plug: A type of device that is inserted in a tear duct (punctum) of an eye. Punctal plugs typically have a conical or cylindrical portion, which is inserted into the punctum, a narrowed neck, and a wider cap that rests on the surface of the punctum; however, variations on the specific components are available. In some examples, a punctal plug occludes the tear duct. In other examples, a punctal plug is non-occlusive (for example, includes a perforated portion to allow tear flow).

Subject: Living multi-cellular vertebrate organisms, a category that includes both human and non-human mammals (such as veterinary subjects or wild animals), for example pigs, mice, rats, rabbits, sheep, horses, cows, dogs, cats, ferrets, hamsters, and non-human primates (e.g., rhesus macaques, cynomolgus macaques, and chimpanzees). In some examples, a subject has keratoconus. In an example, a subject is a human.

Therapeutically effective amount: The amount of an agent, such as copper, that is alone (or in combination with other therapeutic agents) sufficient to prevent, treat (including prophylaxis), reduce and/or ameliorate the symptoms and/or underlying causes of a disease or disorder, for example to prevent, inhibit, and/or treat keratoconus. In some aspects, a therapeutically effective amount is sufficient to reduce or eliminate a symptom of a disease, such as a symptom of keratoconus.

A therapeutically effective amount of an agent can be administered in a single dose, in several doses, or substantially continuously, during a course of treatment. However, the therapeutically effective amount can depend on the subject being treated, the severity and type of the condition being treated, and the manner of administration.

### II. Divalent Metal-Containing Biomaterials

Provided herein are divalent metal-containing biomaterials (such as biocompatible and/or biodegradable materials) for use in treating eye disorders, such as keratoconus. In some examples, the biomaterials include a biodegradable hydrogel and an active agent including a divalent metal, such as a copper salt, magnesium salt, or iron salt.

In some embodiments, the disclosed biomaterials include a hydrogel and an active agent, wherein the active agent is incorporated in or is in association with the hydrogel. As described herein, the biomaterial is prepared such that the active agent is released over time, for example, as the hydrogel biodegrades when implanted in a subject. In some examples, the hydrogel includes a polymer such as polyethylene glycol (PEG), polycaprolactone (PCL), polyoctanediol-co-citrate (POC), polylacto-glycolic acid (PLGA), or polyoxyethylene bis(amine) (PEO). In particular examples, the polymer or pre-polymer includes a diacrylate, such as PEG diacrylate (PEGDA) or PCL diacrylate (PCL-DA). In one specific example, the hydrogel is formed from PEGDA with average Mₙ 700, average Mₙ 500, or average Mₙ 250.

In some embodiments of the disclosed biomaterials, the active agent includes a divalent metal, such as a divalent metal salt, for example, a sulfate, citrate, acetate, nicotinate, carbonate, phosphate, dihydrogen phosphate, asprinate, naproxen, histidine, stearate, oleate, chloride, oxide, lactate, dilactate, hydroxide phosphate, or threonate salt. In some examples, the salt is not a sulfate, carbonate, phosphate, or histidine salt.

In some examples, the salt is a copper salt. In some examples, the copper salt is selected from copper sulfate, copper citrate, copper acetate, copper nicotinate, copper carbonate, copper phosphate, copper dihydrogen phosphate, copper asprinate, copper naproxen, copper histidine, copper stearate, copper oleate, copper chloride, copper oxide, copper lactate, copper dilactate, copper hydroxide phosphate, and copper threonate. In one specific example, the copper salt is copper dilactate. In other examples, the copper salt is not copper sulfate, copper carbonate, copper phosphate, or copper histidine.

In other embodiments, the active ingredient includes a magnesium salt (such as magnesium sulfate, magnesium citrate, magnesium acetate, magnesium nicotinate, magnesium carbonate, magnesium phosphate, magnesium dihydrogen phosphate, magnesium asprinate, magnesium naproxen, magnesium histidine, magnesium stearate, magnesium oleate, magnesium chloride, magnesium oxide, magnesium lactate, magnesium dilactate, magnesium hydroxide phosphate, or magnesium threonate) or a ferrous salt (such as iron sulfate, iron citrate, iron acetate, iron nicotinate, iron carbonate, iron phosphate, iron dihydrogen phosphate, iron asprinate, iron naproxen, iron histidine, iron stearate, iron oleate, iron chloride, iron oxide, iron lactate, iron dilactate, iron hydroxide phosphate, or iron threonate). In some examples, the magnesium salt is not magnesium sulfate, magnesium carbonate, magnesium phosphate, or magnesium histidine. In other examples, the iron salt is not iron sulfate, iron carbonate, iron phosphate, or iron histidine.

In some embodiments, the active agent includes a nanoparticle including the divalent metal. The metal nanoparticles may be copper nanoparticles, iron nanoparticles, or magnesium nanoparticles. In some examples, the metal nanoparticles have a uniform size distribution, such as a particle size of less than about 1 μm, for example, about 10 nm to about 20 nm.

In some embodiments, the biomaterial includes at least about 1 wt %, at least about 5 wt %, at least about 10 wt %, at least about 15 wt %, at least about 20 wt %, at least about 25 wt %, at least about 30 wt %, at least about 35 wt %, or at least about 40 wt % active agent. In some examples, the biomaterial includes about 1 to about 20 wt % active agent (for example, about 1 to about 5 wt %, about 2.5 to about 7.5 wt %, about 5 to about 10 wt %, about 7.5 to about 12.5 wt %, about 10 to about 15 wt %, about 12.5 to about 17.5 wt %, or about 15 to about 20 wt % active agent). In some examples, the biomaterial includes about 7.18 to about 14.36 wt % active ingredient. In one specific example, the biomaterial includes about 7 wt % active agent (such as about 7 wt % copper). In another specific example, the biomaterial includes about 14 wt % active agent (such as about 14 wt % copper).

In other embodiments, the biomaterial includes at least about 20 μg, at least about 50 μg, at least about 75 μg, at least about 100 μg, at least about 125 μg, at least about 150 μg, at least about 175 μg, or at least about 200 μg active agent. In some examples, the biomaterial includes about 20 to about 200 μg active agent (for example, about 20 to about 40 μg, about 30 to about 60 μg, about 50 to about 100 μg, about 75 to about 125 μg, about 100 to about 150 μg, about 125 to about 175 μg, or about 150 to about 200 μg active agent). In some specific examples, the biomaterial includes at least about 35 μg active agent (such as about 35 μg copper), at least about 70 μg active agent (such as about 70 μg copper), at least about 90 μg active agent (such as about 90 μg copper), or at least about 180 μg active agent (such as about 180 μg copper).

The amount of active agent in the biomaterial can be selected based on the size and shape of the biomaterial, as well as the expected degradation pattern of the biomaterial. Thus in some examples, the biomaterial includes an amount of active agent such that about 0.5 to about 1 μg is released per day. In one specific example, a biomaterial can include about 90-180 μg active agent, resulting in release of about 0.5 to about 1 μg active agent per day over 3-6 months. In some examples, the biomaterial includes about 90-180 μg active agent in a 0.3-0.5 mm diameter material.

In some embodiments, the disclosed biomaterials provide for sustained delivery of the active agent to the eye, for example by release of the active agent as the biomaterial degrades or is resorbed. In some examples, the biomaterial provides sustained delivery of the active agent to the eye over a period at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In some examples, the active agent is released from the biomaterial at a rate of about 0.1 μg/day to about 2 μg/day, for example, about 0.1 to about 0.25 μg/day, about 0.25 to about 0.75 μg/day, about 0.5 to about 1.0 μg/day, about 0.75 to about 1.5 μg/day, or about 1.0 to about 2.0 μg/day. In some examples, the active agent is released at about 0.5 μg/day or about 1.0 μg/day.

In some examples, delivery or release of the active agent is substantially continuous. In some examples, the delivery or release of the active agent exhibits linear zero-order release kinetics. In other examples, the delivery or release of the active agent exhibits modified first-order release kinetics (such as a short burst of release followed by slowly declining release over a period of time, such as 2-6 months). In other examples, delivery or release of the active agent may exhibit slow or sustained delivery or release of the active agent over a period of time, followed by a burst of active agent release, for example upon final degradation of the biomaterial.

In some embodiments, the provided biomaterial is in the form of a punctal plug. Punctal plugs can be inserted into the tear duct, for example, for drug delivery for treating keratoconus. In some examples, the punctal plug is occlusive, while in other examples, the punctal plug is non-occlusive. In some examples, the plug has dimensions of about 0.2-0.6 mm diameter (such as about 0.2 mm, about 0.25 mm, about 0.3 mm, about 0.35 mm, about 0.4 mm, about 0.45 mm, about 0.5 mm, about 0.55 mm, or about 0.6 mm diameter). In some examples, the plug is and about 2-5 mm long (such as about 2 mm, about 2.5 mm, about 3 mm, about 3.5 mm, about 4 mm, about 4.5 mm, or about 5 mm long). In some examples, the punctal plug includes additional portions, such as a narrowed neck attached to the plug, and optionally a wider cap that rest on the surface of the punctum. However, one of ordinary skill in the art can select other dimensions and components, for example, based upon the subject being treated.

In other embodiments, the biomaterials are provided in the form of an implant (such as a thin film, a flat circle or disc, a rod, or a spherical shell) that can be inserted into a conjunctival fornix (such as a lower conjunctival fornix). One of ordinary skill in the art can select appropriate dimensions, for example, based upon the subject being treated. Exemplary dimensions for human and rabbit lower fornix are shown in Table 1.

In some embodiments, a conjunctival implant or insert for use in a human subject is about 8 mm to about 15 mm long (such as about 8 mm, about 9 mm, about 10 mm, about 11 mm, about 12 mm, about 13 mm, about 14 mm, or about 15 mm long), about 4 mm to about 6 mm high (such as about 4 mm, about 4.5 mm, about 5 mm, about 5.5 mm, or about 6 mm high), and about 0.05 mm to about 0.5 mm thick (such as about 0.05 mm, about 0.1 mm, about 0.15 mm, about 0.2 mm, about 0.25 mm, about 0.3 mm, about 0.35 mm, about 0.4 mm, about 0.45 mm, or about 0.5 mm thick). In other examples, the conjunctival implant or insert is a rod, for example with a diameter of about 0.5 mm to about 1.5 mm (such as about 0.5 mm, about 0.6 mm, about 0.7 mm, about 0.8 mm, about 0.9 mm, about 1 mm, about 1.1 mm, about 1.2 mm, about 1.3 mm, about 1.4 mm, or about 1.5 mm in diameter) and about 3 mm to about 10 mm long (such as about 3 mm, about 4 mm, about 5 mm, about 6 mm, about 7 mm, about 8 mm, about 9 mm, or about 10 mm long). The volume of a conjunctival inserts may be about 0.7 μl to about 28 μl (such as about 0.7 to about 2 μl, about 1 to about 3 μl, about 2.5 to about 5 μl, about 5 to about 10 μl, about 10 to about 15 μl, about 15 to about 20 μl, or about 20 to about 28 μl). In another example, a disclosed film for use in rabbits has dimensions of about 15 mm long x about 2-3 mm wide x about 0.5-1 mm thick.

TABLE 1
Exemplary lower fornix dimensions

| Species | Dimensions (mm) | Reference | Notes |
|---|---|---|---|
| Human | 11.9 - 40-49 Y | Schwab et al, | Mean depth |
| | 11.3 - 50-59 Y | Ophthalmology | measurements |
| | 11.0 - 60-69 Y | 99: 197-202, | |
| | 10.6 - 10.6 Y | 1992 | |
| | 10.2 - 80+ | | |
| | 11.1-9.6 - F 20-80 Y | Jutley et al., | Mean depth |
| | 11.4-10.8 - M 20-80 Y | Eye (Lond.) | measurements |
| | | 30: 1351-1358, | (British |
| | | 2016 | Caucasian) |
| | 10.2 - Depth | Kawakita et al., | Sample details: |
| | 399.6 ± 102.8 mm² - | Eye (Lond.) | 13 women, 7 men |
| | Area | 23: 1115-1119, | (Japanese); mean |
| | | 2009 | age: 64.1 ± 9.6 |
| | | | years, range: 38-80 |
| | | | years |
| | 11.3-9.9 F 20-70 Y - | Khan et al., | South Asian Origin |
| | Depth | Ophthalmology | population in UK |
| | 11.9-10.5 - M 20-70 | 121: 492-497, | |
| | Y - Depth | 2014 | |
| | 33.6-28.5 F 20-70 Y - | | |
| | Intercanthal distance | | |
| | 33.3-30.1 M 20-70 Y - | | |
| | Intercanthal distance | | |
| Rabbit | 4 mm wide by 15 mm | Griffith et al., | Based on |
| | long film | Burns 44: 1179- | CMHA-S film |
| | | 1186, 2018 | placed in lower |
| | | | fornix |
| | Hydrogel geometry | Colter et al., | Testing geometry |
| | 0.88-3 mm thickness | Ann. Biomed. | of hydrogels in |
| | 15-17.6 length | Eng. 46: 211- | lower fornix |
| | 1.5-3 - width | 221, 2018 | |
| | 32 mm² area strips of | Rubin et al, J. | Collagen |
| | film used to place in | Clin. Pharmacol. | membrane cut |
| | lower fornix 0.46 mm | 13: 309-312, | into strips |
| | thickness/depth | 1973 | |

### III. Methods of Making Divalent Metal-Containing Biomaterials

Methods of making the disclosed divalent metal-containing biomaterials are provided. In some embodiments, the methods include incorporating a divalent metal salt (such as a copper salt, magnesium salt, or iron salt) into a hydrogel material.

In some embodiments, the methods include mixing a solution of pre-polymer with a divalent metal salt solution and crosslinking or curing the polymer to form a hydrogel material in association with the divalent metal salt. In some examples, the methods include mixing a solution of pre-polymer (such as PEG, PCL, POC, PLGA, or PEO) with a divalent metal salt solution, sonicating the mixture of pre-polymer and divalent metal salt solution, adding a photoinitiator, and curing under UV light to form the divalent metal-containing material. In some examples, adding a photoinitiator and UV curing may not be required, depending on the polymer hydrogel. In some examples, the mixture of pre-polymer, divalent metal salt solution, and photoinitiator is placed in a mold of a desired size and shape prior to curing. In some examples, the pre-polymer is PEG-DA or PCL-DA. In some examples, the mixture of pre-polymer and divalent metal salt solution is sonicated for about 5 to about 15 minutes. In some examples, the sonicator is a bath sonicator or a probe sonicator. In some examples, the sonicator amplitude is about 20 kHz to about 40 kHz (such as about 20 kHz, about 25 kHz, about 30 kHz, about 35 kHz, or about 40 kHz).

In some embodiments, the divalent metal salt solution is prepared by sonicating a solution of the divalent metal salt, incubating the sonicated mixture at 37° C., and cooling the solution to room temperature. In some examples, the divalent metal salt solution is sonicated for a total of about 30 to about 120 minutes. The sonication may be using a bath sonicator in 2-8 periods of about 5 to 15 minutes, with a rest period between sonication (for example of about 10 to 15 minutes at room temperature). Thus in some examples, the solution of divalent metal salt is sonicated for about 15 minutes, about 30 minutes, about 45 minutes, about 60 minutes, about 75 minutes, about 90 minutes, about 105 minutes, or about 120 minutes total. In other examples, the sonication may be using a probe sonicator in periods of about 2 to about 20 seconds (such as about 2 seconds, about 5 seconds, about 10 seconds, about 15 seconds, or about 20 seconds) with a rest period between sonication (for example, of about 30 seconds to about 2 minutes on ice).

Following sonication, the sonicated solution may be incubated at 37° C. for a period of time before mixing with the pre-polymer solution. In some examples, the sonicated solution is incubated at 37° C. for about 6 hours to about 24 hours (for example, about 6-12 hours, about 8-16 hours, about 12-18 hours, or about 16-24 hours). In one example, the sonicated divalent metal salt solution is incubated at 37° C. overnight prior to mixing with the pre-polymer solution.

In some embodiments, the divalent metal salt solution is a copper salt solution, a magnesium salt solution, or an iron salt solution. In some examples, the copper salt is selected from the group of copper sulfate, copper citrate, copper acetate, copper nicotinate, copper carbonate, copper phosphate, copper dihydrogen phosphate, copper asprinate, copper naproxen, copper histidine, copper stearate, copper oleate, copper chloride, copper oxide, copper lactate, copper dilactate, copper hydroxide phosphate, and copper threonate. In one specific example, the copper salt is copper dilactate.

In other embodiments, the divalent metal is in the form of metal nanoparticles (such as copper nanoparticles) and is incorporated into the hydrogel. In some examples, the metal nanoparticles are formed in situ during preparation of the hydrogel, for example as a result of the UV curing step of the disclosed methods.

### IV. Methods of Treatment

Provided herein are methods of treating an eye disease or disorder with a disclosed divalent metal salt-containing biomaterial. In some examples, the methods include treating keratoconus in a subject. In other examples, the methods include treating a subject with myopia (such as pediatric myopia or adult myopia), astigmatism, pellucid marginal degeneration, presbyopia, corneal ulcers, corneal marginal degenerations, astigmatism, scleral melting (scleromalacia) and corneal ectasia.

In some embodiments, the methods include contacting the eye of a subject with an eye disease or disorder with a disclosed biocompatible material. In one embodiment, the biocompatible material is in the form of a punctal plug, and the plug is placed in a tear duct of the subject with the eye disease or disorder. In other examples, the biocompatible material is in the form of a strip, film, or rod, and the material is placed in contact with a fornix of the subject with keratoconus (such as the lower fornix). In some examples, the methods include contacting one eye of the subject with a disclosed biocompatible material (e.g., unilateral treatment). In other examples, the methods include contacting both eyes of the subject with a disclosed biocompatible material (e.g., bilateral treatment).

In some examples, the subject is treated one time, for example with a single punctal plug or fornix implant. In other examples, the subject is treated two or more times at intervals of at least about 3 months, at least about 6 months, at least about 9 months, or at least about 12 months. In some examples, the subject is assessed periodically for presence of the punctal plug or implant, and may receive a second or further treatment following degradation of the punctal plug or implant. In other examples, the subject is assessed for symptoms of keratoconus and is administered a second or subsequent treatment if the symptoms are unchanged or worsened compared to prior to the treatment. The number of treatments can be selected by a skilled clinician, based on factors such as the severity of the condition being treated and the age of the patient. For example, keratoconus typically stops progressing around age 40.

In some embodiments, the subject may be treated with one or more additional therapies for the eye disease or disorder. In some examples, the additional therapy is not included in the biocompatible material disclosed herein, for example, the additional therapy is a separate treatment or composition. For example, for keratoconus, the subject may further be treated with riboflavin, rose Bengal, or hydroxylysine. The subject may also receive glasses, soft contact lenses, toric or hard contact lenses, intrastromal corneal rings, corneal collagen cross-link therapy, deep anterior lamellar keratoplasty, or penetrating corneal transplant. One of ordinary skill in the art can identify appropriate additional therapies based on the eye disease or disorder being treated, the severity of the condition, the age of the patient, and other factors.

In some embodiments, the disclosed methods of treatment improve one or more signs or symptoms of the eye disease or disorder. For example, the methods described herein result in improvement in one or more symptoms of the eye disease or disorder compared to a control, such as an untreated subject with the disorder, or an untreated eye in the subject with the disorder. In other examples, the methods described herein result in slowed disease progression, for example, compared to an untreated subject or untreated eye of the subject.

In some examples, the subject has keratoconus. Symptoms of keratoconus can be assessed by one or more of corneal thickness, inter-eye asymmetry, inferior-superior asymmetry, maximum keratometry, and corneal hysteresis. In some examples, the methods described herein result in increased corneal crosslinking, increase lysylnorleucine (a marker of LOX activity), reduction in maximum keratometry, decline in mean corneal astigmatism, increased stiffness parameter highest curvature (SP-HC), and increased stress-strain index.

### EXAMPLES

The following examples are provided to illustrate particular features of certain aspects of the disclosure, but the scope of the disclosure should not be limited to those features exemplified.

### Example 1

### Poly Octanediol-Co-Citrate Plugs

Equimolar amounts of citric acid and 1,8-octanediol were mixed and melted at 160-165° C. under a flow of nitrogen gas with stirring. Upon melting, the temperature was lowered to 140° C. and maintained for 30 min with continuous stirring to create a pre-polymer. The pre-polymer was subject to temperatures of 60, 80, or 120° C. under vacuum (2 Pa) or no vacuum for times ranging from 1 day to 2 weeks to enable polymerization, resulting in POC with various degrees of cross-linking. In order to obtain punctal plugs of desired dimensions of 300 μm diameter cylinders, POC was molded in polytetrafluoroethylene (PTFE) tubing (AWG 30). After polymerization, the tubing was cut and peeled off to obtain cylinders of dimension 305 μm and 2-4 mm length. In order to add copper to the POC plugs, a copper sulfate solution was prepared by adding 0.008 grams of CuSO₄ to 20 mL methanol. In order to make the POC/copper plugs, pre-cured and molded POC of the desired dimension was soaked in a solution of 2 mM copper sulfate. The pre made POC plugs were soaked in the solution for approximately 6 hours. For testing degradation, POC plugs were soaked in BSS solution at 37° C. and were completely degraded in 5-7 days.

### Example 2

### Polycaprolactone Plugs

In order to make PCL/copper plugs, the solvent displacement method was experimented with to load a polar salt, copper sulfate, into the non-polar matrix of PCL. The method was tested initially with copper sulfate and then switched to cupric dilactate. PCL (10 grams) was solubilized in tetrahydrofuran (THF, 20 grams), and copper sulfate or cupric dilactate (0.04-1 gram) was prepared in water (20 mL). The copper solution (20 mL) was then added to the PCL solution (20 mL) while heating (80° C.) and stirring for approximately 24 hours. The molten PCL containing copper was molded into PTFE tubing and allowed to solidify. In order to remove the implant from the tubing, the tubing was cut with a razor blade and peeled off of the PCL. However, it was difficult to incorporate copper sulfate and cupric dilactate (highly non-polar) with PCL in THF. When examined under the microscope, the copper (blue/green color) was very unevenly distributed. Also, the copper loading was significantly below the desired amounts by inductively coupled plasma mass spectrometry (ICP-MS) analysis (Table 2).

TABLE 2
Copper content of native PCL polymer
and copper-loaded PCL plugs by ICP-MS

| | Length | Diameter | Mass | Copper | Copper |
|---|---|---|---|---|---|
| Group | (mm) | (mm) | (mg) | (ng)* | (ng/mg) |
| Control PCL native | 9.3 | 1.72 | 29.4 | ND | |
| Cu + PCL-1 | 4.93 | 0.36 | 0.45 | 333.1 | 740.2 |
| Cu + PCL-2 | 3.9 | 0.36 | 0.34 | 288.68 | 849.1 |
| Cu + PCL-3 | 7.5 | 0.36 | 0.70 | 528.2 | 754.6 |

*Mean of five readings.
ND = not detected

### Example 3

### Polyethylene Glycol Plugs

Polyethylene glycol diacrylate (PEGDA) Mn700 in a liquid form was mixed with cupric dilactate solution, equal volumes, vortexed, and sonicated for 5 minutes. Visual examination indicated good miscibility. TPO (Diphenyl(2,4,6-trimethylbenzoyl)phosphine oxide) photoinitiator (1-2%) was added to the mix, sonicated, and filtered through a 0.45 μM syringe filter. The mixture thus obtained was cured under UV light for 30-60 minutes in PTFE molds with an internal diameter of 300-500 μm. These were measured for copper content by ICP-MS (Table 3). The PEGDA plugs had more than 10-fold increased copper content compared to PCL (ng/mg).

TABLE 3
Copper content of control and copper-
loaded PEGDA plugs by ICP-MS

| | Length | Diameter | Mass | Copper | Copper |
|---|---|---|---|---|---|
| Group | (mm) | (mm) | (mg) | (μg)* | (ng/mg) |
| Control PEGDA | 10 | 1.72 | 3.69 | ND | |
| Cu + PEGDA -1 | 5 | 0.3 | 1.15 | 16.2 | 14,087 |
| Cu + PEGDA -2 | 4.5 | 0.3 | 1.03 | 15.4 | 14,951 |
| Cu + PEGDA -3 | 3.16 | 0.3 | 0.51 | 7.1 | 13,921 |

*Mean of five readings.
ND = not detected

### Example 4

### Increasing Cupric Dilactate Loading

Increased concentrations of cupric dilactate were prepared for use in polymers to achieve higher loading. Excess of cupric dilactate in water (5 g in 10 ml) was prepared and sonicated for 30-120 minutes (15 minutes each x 2-8 times with 10 minutes break between each sonication session) and left overnight at 37° C. The solution obtained was cooled to room temperature and the green liquid containing cupric dilactate was separated from the undissolved cupric dilactate by aspiration and filtering through 0.45 μm syringe filter. No precipitate was observed in the filtrate over 3 weeks. This solution can be used for loading plugs, for example as described in Examples 1-3.

Alternatively, cupric dilactate may be dissolved in hot water (60-80° C.), sonicated, and incubated overnight at 37° C., to potentially increase the amount of cupric dilactate in the same volume of water.

### Example 5

### In Vitro Testing of Copper-Loaded Implants

Stromal cells are removed from donor corneas and mechanically separated from the epithelium to obtain corneal fibroblasts for culture. Descemet's membrane is removed mechanically under a stereo microscope before cutting the cornea into small pieces for digestion with 10 ml of 1 mg/ml collagenase1A-S(Sigma-Aldrich). After filtering through a 40 μm cell strainer, the corneal stromal cells are suspended in 15% fetal bovine serum (FBS) in DMEM/F12 with penicillin/streptomycin. Normal and keratoconic human fibroblast cultures are incubated with copper-loaded implants or non-loaded implants for 5 days. The supernatant/media is analyzed by ICP-MS for copper content and used for LOX assays and estimation of LOX, MMPs, and TIMPs by ELISA. Cells are harvested and used for protein expression of MMP-2/9, TIMP-1/2, and caspases 3/7 by Western blotting. Cells from a separate set of experiments are used for TUNEL assay and crystal violet staining to confirm that the implant does not have an adverse effect on cell confluence or apoptosis. Cells are also analyzed for copper (uptake) by ICP-MS. An increase in LOX activity and decrease in MMP-2/9 is expected in the media of cells incubated with copper-loaded implants.

### Example 6

### In Vivo Testing of Copper-Loaded Implants

New Zealand white rabbits are implanted with copper-loaded punctal plugs (2 mm long, 0.2 mm diameter to fit within rabbit lacrimal ducts) in one eye; the other untreated eye will serve as a control. Tears are collected from rabbit eyes for baseline analysis of copper by ICP-MS, LOX activity, MMPs, and TIMPs. Thereafter, implants are placed in one eye of each rabbit under anesthesia. Subsequently, tears are collected from each eye of every rabbit at 2-week intervals for 6 months and analyzed for copper content by ICP-MS, while LOX, MMP-2/9, and TIMPs-1/2 are measured by fluorometry and ELISA from monthly samples. Rabbits receive monthly eye exams to ensure that the implants do not have an adverse effect on surrounding tissue. Rabbits undergo monthly corneal topography (e.g., with the Optikon Keratron Scout). Brillouin optical spectroscopy scanner (Intelon; Lexington, Mass.) is used to serially assess the biomechanical properties (longitudinal bulk modulus; stromal elastic modulus) of live rabbit corneas with three-dimensional resolution. This method measures viscoelasticity and corneal stiffness by probing the hypersonic acoustic waves intrinsic to the cornea. It is expected that biomechanical strength will increase over time in eyes treated with copper-loaded plugs, and that this will correlate with increased LOX activity, increased TIMP-1/2, and decrease MMP-2/9.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

We claim:

1. A biocompatible material comprising:
a biodegradable hydrogel; and
a therapeutically effective amount of an active agent comprising a divalent metal salt, wherein the active agent is incorporated in the hydrogel.

2. The biocompatible material of claim 1, wherein the biodegradable hydrogel comprises polyethylene glycol or polycaprolactone.

3. The biocompatible material of claim 2, wherein the biodegradable hydrogel comprises polyethylene glycol diacrylate or polycaprolactone diacrylate.

4. The biocompatible material of claim 1, wherein the divalent metal salt is a copper salt, a magnesium salt, or an iron salt.

5. The biocompatible material of claim 4, wherein the copper salt is selected from the group of copper sulfate, copper citrate, copper acetate, copper nicotinate, copper carbonate, copper phosphate, dicopper hydrogen phosphate, copper asprinate, copper naproxen, copper histidine, copper stearate, copper oleate, copper chloride, copper oxide, copper lactate, copper dilactate, and copper threonate.

6. The biocompatible material of claim 4, wherein the material comprises about 1 to about 20 wt % copper.

7. The biocompatible material of claim 4, wherein about 0.5 μg to about 1 μg of copper is released per day.

8. The biocompatible material of claim 1, wherein the material is formed as a punctal plug or a film.

9. The biocompatible material of claim 1, wherein the material biodegrades or resorbs over at least 3 months when implanted in an eye.

10. A method of making the biocompatible material of claim 1, comprising:
mixing pre-polymer with a divalent metal salt solution;
sonicating the mixture of pre-polymer and divalent metal salt solution;
adding a photoinitiator; and
curing under UV light to form a divalent metal salt-containing material.

11. The method of claim 10, wherein the divalent metal salt solution is prepared by:
sonicating a solution of the divalent metal salt;
incubating the sonicated solution at 37° C.; and
cooling the solution to room temperature.

12. The method of claim 11, wherein the solution of divalent metal salt is sonicated for about 30-120 minutes.

13. The method of claim 12, wherein the sonicated solution is incubated at 37° C. for about 6 to about 24 hours.

14. The method of claim 10, wherein the pre-polymer is polyethylene glycol diacrylate or polycaprolactone diacrylate.

15. The method of claim 10, wherein the divalent metal salt solution is a copper salt solution.

16. The method of claim 15, wherein the copper salt is selected from the group of copper sulfate, copper citrate, copper acetate, copper nicotinate, copper carbonate, copper phosphate, copper dihydrogen phosphate, copper asprinate, copper naproxen, copper histidine, copper stearate, copper oleate, copper chloride, copper oxide, copper lactate, copper dilactate, copper hydroxide phosphate, and copper threonate.

17. A method of treating an eye disease or disorder in a subject, comprising contacting the eye of the subject with the biocompatible material of claim 1.

18. The method of claim 17, wherein the biocompatible material is placed in contact with a tear duct or fornix of the subject with keratoconus.

19. The method of claim 17, wherein the eye disease or disorder comprises keratoconus, myopia, presbyopia, corneal ulcers, corneal marginal degenerations, astigmatism, scleromalacia, or corneal ectasia.

20. The method of claim 17, further comprising administering to the subject one or more additional therapies for the eye disease or disorder.
